(19) United States
(12) Patent Application Publication
(10) Pub. No.: US 2019/0122753 A1
(43) Pub. Date: Apr. 25, 2019

(54) METHOD, APPARATUS AND SYSTEM FOR RENDERING AND DISPLAYING MEDICAL IMAGES

(71) Applicant: Siemens Aktiengesellschaft, Muenchen (DE)

(72) Inventors: Thomas FRIESE, Munich (DE); Thomas GOSSLER, Erlangen (DE)

(73) Assignee: Siemens Aktiengesellschaft, Muenchen (DE)

(21) Appl. No.: 16/185,245

(22) Filed: Nov. 9, 2018

Related U.S. Application Data
(62) Division of application No. 14/362,504, filed on Jun. 3, 2014, now abandoned.

(30) Foreign Application Priority Data
Feb. 22, 2012 (DE) 102012202701.7

(51) Int. Cl. G16H 10/60; G06Q 10/10

(52) U.S. Cl. CPC G16H 10/60; G06F 19/00; G06Q 10/10

(57) ABSTRACT

An image acquisition and processing method includes: scanning a patient to acquire raw data for a selected patient; generating medical image data based on the raw data; storing the medical image data along with sensitive patient data for the selected patient in an anonymized patient-related data record; rendering the medical image data stored in the anonymized patient-related data record to generate a rendered medical image for display in response to a received query relating to the sensitive patient data; displaying the rendered medical image in response to determining that verification data matches query data generated by applying a one-way hash function to a received query; and displaying a fault notification in response to determining that the verification data does not match the query data.

### CROSS-REFERENCE TO RELATED APPLICATIONS

This application is a Divisional Application of, and claims priority under 35 U.S.C. 120/121 to, U.S. application Ser. No. 14/362,504, filed Jun. 3, 2014, which is the U.S. National Phase under 35 U.S.C. § 371 of PCT International Application No. PCT/EP2012/074334, filed Dec. 4, 2012, which designated the United States of America, and which claims priority to German patent application No. DE 10 2012 202 701.7, filed Feb. 22, 2012.

### FIELD

At least one embodiment of the invention generally relates to a method, apparatus and/or system for rendering and displaying medical images.

### BACKGROUND

Current developments in the medical sector are aimed at providing a central information technology system which can be used to collate and archive the medical data relating to each patient in such a manner that each doctor determined by the patient is able to easily and quickly access all medical data relating to the patient which are required by the doctor.

For this purpose, it is necessary to transfer medical data relating to the patient from the immediate control area of individual medical facilities to a cloud computing architecture jointly used by a plurality of users. In this case, it is desirable, or often also necessary on account of legal provisions, to remove the so-called “Protected Health Information” (PHI), that is to say all data which make it possible to uniquely identify the patient, from the medical data relating to the patient. This also applies, for example, to data which have been removed according to the DICOM (Digital Imaging and Communications in Medicine) standard and contain image data which are created, for example, during examinations using a computer tomograph. The “Protected Health Information” can also be anonymized in this case by allocating a pseudonym, for example, provided that the pseudonym is known only to the originator of the data, that is to say the respective medical facility.

In order to ensure patient safety and, in particular, to avoid misdiagnoses, there is also the requirement, when generating image data as part of an examination using an image-generating medical system, for the patient identity to be inextricably linked to the generated image data, with the result that incorrect assignment of image data to a patient is excluded as far as possible.

On account of these two contradictory requirements, the use of cloud computing architectures which are jointly used by a multiplicity of users has previously usually been dispensed with or else the cloud computing architecture was located, together with all access operations, in the control area of an individual medical facility since, in this case, there is no need to anonymize the “Protected Health Information”. In another frequently used solution, only encrypted data are delivered to the cloud computing architecture and are made available in the latter, in which case the data can be decrypted using a client application locally installed with the user. Depending on the volume of data and type of encryption, a very large amount of computational complexity is associated with corresponding encryption of the data or decryption of the data. Since the data must generally be present in decrypted form for further processing, it is also necessary to respectively transmit the entire data record in this case. Therefore, this solution is disadvantageous, in particular, in the case of image data and/or in the case of user access operations in which there is locally only relatively little computational power and/or in networks in which some network connections have a relatively narrow bandwidth for data transmission.

### SUMMARY

At least one embodiment of the invention specifies an alternative and advantageous method for rendering and displaying medical images.

A method is disclosed. The dependent claims comprise in part advantageous and in part inherently inventive developments of this invention.

The method of at least one embodiment is used to render and display medical images. During the method, the sensitive patient data in each patient-related data record are anonymized, thus producing anonymized patient-related data records. Furthermore, test data are generated from the respective sensitive patient data in each patient-related data record with the aid of an algorithm and are incorporated in the respective patient-related data record. The anonymized patient-related data records containing the test data are then provided in a cloud computing architecture.

### BRIEF DESCRIPTION OF THE DRAWINGS

Example embodiments of the invention are explained in more detail below using a schematic drawing, in which:

FIG. 1 shows a block diagram of a method for rendering and displaying medical images.

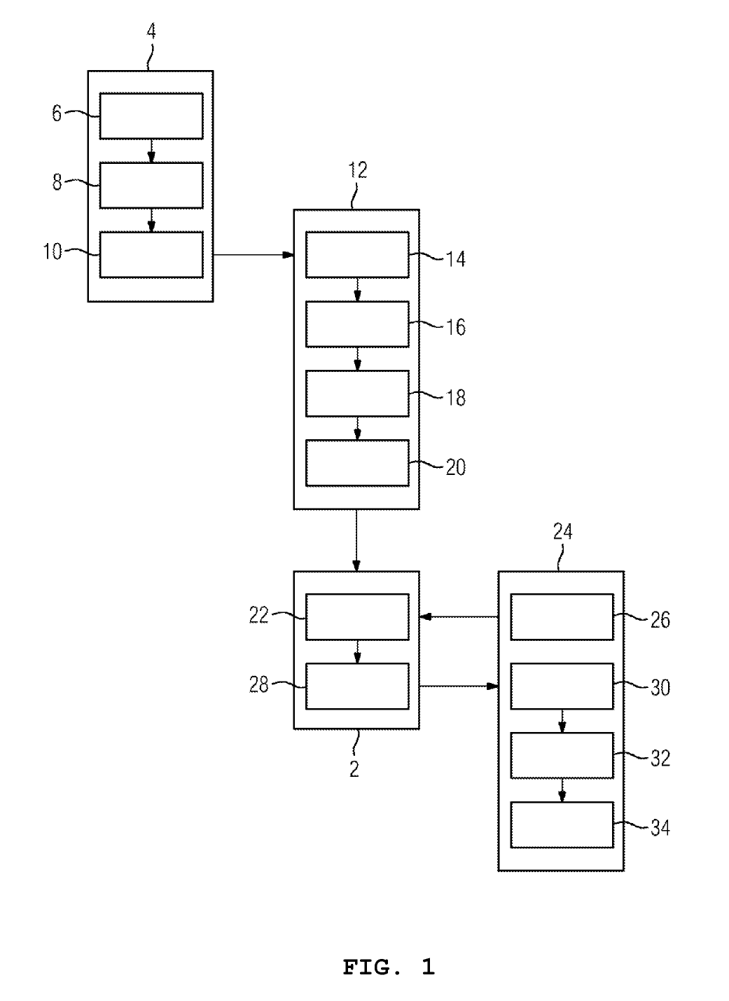

### DETAILED DESCRIPTION OF THE EXAMPLE EMBODIMENTS

The method of at least one embodiment is used to render and display medical images. During the method, the sensitive patient data in each patient-related data record are anonymized, thus producing anonymized patient-related data records. Furthermore, test data are generated from the respective sensitive patient data in each patient-related data record with the aid of an algorithm and are incorporated in the respective patient-related data record. The anonymized patient-related data records containing the test data are then provided in a cloud computing architecture.

In addition, sensitive patient data relating to a selected patient are predefined on a client computer, which is connected to the cloud computing architecture, during processing of a particular patient-related data record, and query data are generated from these predefined sensitive patient data with the aid of the algorithm. A security function is triggered if the query data relating to the selected patient do not match the test data in the particular patient-related data record. In this case, the expression “patient-related data records” represents, in particular, files according to the DICOM (Digital Imaging and Communications in Medicine) standard and the expression “sensitive patient data” comprises, in particular, so-called “Protected Health Information” (PHI).

The complete patient-related data records are therefore not encrypted in this method, but rather only individual items of information contained therein, namely the sensitive patient data, are concealed. This is effected, for example, by encrypting the sensitive patient data, such as the patient's name, the patient's date of birth etc., in a manner in which the corresponding plain data are replaced with suitable placeholders. Consequently, the patient-related data records can be processed further even after the sensitive patient data have been anonymized without having to previously reverse the anonymization of the sensitive patient data.

Accordingly, the anonymized patient-related data records can be provided in the cloud computing architecture and can be stored and/or processed further in the latter without the sensitive patient data appearing as plain data within the cloud computing architecture. In addition, the sensitive patient data, even if anonymized, permanently remain incorporated in the patient-related data records, with the result that the two contradictory requirements mentioned at the outset are met in this method. Only authorized persons, in particular the doctors who are selected by the respective patient, are aware of the sensitive patient data as plain data and have access to an application which can be used by the doctors to generate the anonymized sensitive patient data, that is to say the placeholders in particular, from the plain data on a client computer, are given access to the patient-related data records.

The authorized persons are then given access to the patient-related data records via this client computer which is connected to the cloud computing architecture. Since only a comparison is carried out here, in which the anonymized sensitive patient data generated on the client computer are compared with the anonymized sensitive patient data in the anonymized patient-related data records, the plain data also do not appear in the cloud computing architecture even when accessing the latter.

For the benefit of data processing which is as simple as possible, the anonymized sensitive patient data, that is to say the placeholders in particular, are additionally used to form an additional so-called “tag” and the corresponding “tag” is incorporated in the corresponding patient-related data record in order to virtually provide the latter with an identification for archiving. “Tag” is generally understood as meaning an item of additional information added to the data record.

In an advantageous development, the sensitive patient data in each patient-related data record are first of all divided into key data and other sensitive patient data, and all sensitive patient data in each patient-related data record are then anonymized, thus producing anonymized patient-related data records. However, test data are generated only from the respective key data in each patient-related data record with the aid of the algorithm and are incorporated in the respective patient-related data record. The anonymized patient-related data records containing the test data are then provided in the cloud computing architecture. Key data relating to a selected patient are predefined on the client computer, which is connected to the cloud computing architecture, during processing of a particular patient-related data record, and query data are generated from these predefined key data with the aid of the algorithm. The security function is consequently triggered if these query data relating to the selected patient do not match the test data in the particular patient-related data record.

This method variant is intended to allow, in particular, simple dealing with the solution presented here. In this case, it is necessary to take into consideration that the sensitive patient data may sometimes contain very large quantities of information, whereas a small subquantity is already generally sufficient to uniquely identify the corresponding patient. Provision is therefore made, for example, for a doctor wishing to retrieve the medical data relating to his patient to be requested by an application on his computer to enter the name and date of birth of his patient in an input window and for these data to then act as key data. Other sensitive patient data which are often likewise included in the patient-related data records, for example the patient's gender, address, health insurance number etc., must neither be known to the doctor nor entered via an input window. Therefore, the other sensitive patient data play no role, in particular, in identifying the patient-related data records, but are likewise anonymized before the corresponding data records are provided in the cloud computing architecture.

A method variant in which the algorithm is given by a one-way hash function, also called a hash algorithm or hash function, is also preferred. In addition, the same algorithm, in particular the same one-way hash function, is preferably used to anonymize the sensitive patient data and to generate the test data. One-way hash functions suitable for cryptography are well known to a person skilled in the art, with the result that a one-way hash function with favorable properties can be readily found. In this case, one-way hash functions of the type MD5, SHA1 or SHA2 are advantageous, in particular.

A method variant in which a number of the anonymized patient-related data records containing the test data from the cloud computing architecture contain display data for display on the client computer is also expedient. A method variant in which a number of the patient-related data records contain image data from an image-generating modality and in which display data for display on the client computer are generated from the image data in one of these patient-related data records in the cloud computing architecture is likewise expedient. This means that image data, for example, which are generated on a computer tomograph during an examination of a patient are likewise available to every doctor having access, via a computer, to the collected medical documents relating to his patient which are provided via the cloud computing architecture.

In this case, provision is made, in particular, for the image data to be processed with the aid of powerful resources within the cloud computing architecture and for only display data to be sent to the client computer, that is to say the computer belonging to the doctor, which display data are then displayed without further processing on the display device, that is to say a monitor for example. Virtually completed images are therefore sent to the computer belonging to the doctor, which images are then only displayed for the doctor. In contrast, the computation-intensive preprocessing of the data generated by the computer tomograph and, in particular, the calculation of 3-D images are carried out in the cloud computing architecture.

The data volume of such completed images which are then sent to the computer belonging to the doctor is also relatively low. Whereas so-called “volume rendering”, for example, that is to say for example processing of the data relating to the entire examined volume of the patient which are generated by the computer tomograph, is carried out in the cloud computing architecture, only a completed image of an individual view of the volume, as selected by the doctor, or of an individual sectional illustration is sent to the computer belonging to the doctor. Therefore, a relatively narrow bandwidth is sufficient to transmit these data and to connect the computer belonging to the doctor to the network.

In addition, a method variant in which the display data and the test data in a particular anonymized patient-related data record are first of all provided on the client computer, in which these test data are then compared with the query data, and in which the security function is triggered if the test data do not match the query data is preferred. The comparison of the data or the testing process is therefore preferably fully carried out locally on the client computer. In this case, this testing process is preferably implemented by a separate application which is therefore entirely separate from the processing of the anonymized patient-related data records, thus ensuring the desired strict separation between the anonymized patient-related data records and the plain data.

In addition, a method variant in which the test data are graphically incorporated in the display data and also incorporated in the manner of a 2-D barcode is advantageous. If, for example, an x-ray of the patient is thus provided via the cloud computing architecture and is only displayed on the monitor of the computer belonging to the doctor, the depiction of a barcode or a QR code, which represents the anonymized sensitive patient data and, in particular, the key data, is situated, for example, in a predefined area of the displayed image, for example in the top right-hand corner. A query process (part of the method) which is suitable in this case is then as follows, for example.

The doctor first of all inputs the name and date of birth of his patient in an input window, whereupon a QR code is generated on the basis of the name and date of birth using a given one-way hash function. A numerical code is additionally generated with the aid of a second one-way hash function. A file in which the same numerical code is incorporated as a “tag” is then called up in the cloud computing architecture. The image data from this file are then processed, thus generating a set of display data. The display data are then sent to the computer belonging to the doctor, these display data likewise containing a QR code.

The testing process is then started, in which the QR code from the display and the QR code generated on the computer belonging to the doctor are virtually optically compared with one another, preferably in a software-based manner. If the two QR codes match, the display data are displayed as an image on the monitor of the computer belonging to the doctor. A second image in which the plain data represented by the QR code, that is to say the patient's name and date of birth, are displayed is then preferably superimposed on said image in the region of the displayed QR code. The doctor therefore does not see an x-ray, in the top right-hand corner of which a QR code is depicted, but rather sees an x-ray, in the top right-hand corner of which the patient's name and date of birth can be seen and read. In contrast, if the two QR codes do not match, the security function is triggered and a fault message is displayed, for example.

In addition, a method variant in which display of the display data is prevented if the security function is triggered is advantageous. If the test data and the query data therefore do not match, the display data are not displayed for the doctor and therefore cannot be seen. If an x-ray of a patient is thus stored, for example, virtually in a patient file belonging to another patient in the cloud computing architecture and if a doctor now attempts to examine the medical documents in this patient file, the doctor will receive, when attempting to look at the x-ray, a warning message stating that the x-ray is not an x-ray of his patient and the x-ray is not displayed.

The method variant described by way of example below allows an archive for medical data to be located outside the immediate control area of a medical facility, here a hospital. In this case, this archive is distributed among a plurality of PACS (Picture Archiving and Communication System) servers which are part of a cloud computing architecture 2.

If a patient is now intended to be examined in the hospital with the aid of a computer tomograph 4, for example, some sensitive patient data, for example the patient's name and date of birth, are first of all stored in a memory of the computer tomograph 4 during an input process step 6 before the examination. The actual examination of the patient is then carried out, during which raw data are generated using the computer tomograph 4 during a scanning process step 8. Once this scanning process step 8 has been concluded, a patient-related data record is created from the raw data, in which data record the sensitive patient data input in the input process step 6 are incorporated during an embedding process step 10. These sensitive patient data are also supplemented with further sensitive patient data which characterize and uniquely identify the examination carried out on the computer tomograph 4. These are, for example, the date and time of the examination, the examination mode, the radiation dose to which the patient was exposed etc. This patient-related data record is then transmitted to a server station 12 within the immediate control area of the hospital.

The raw data in the patient-related data record are further processed in the server station 12 and, during an image process step 14, are converted into image data, more precisely into so-called transverse slices. The patient-related data record processed in this manner is then stored as a copy in the server station 12 and is additionally preprocessed for storage in the archive for medical data outside the immediate control area of the hospital, that is to say in the cloud computing architecture 2.

An additional “tag” containing a numerical sequence or character string as test data is incorporated in the patient-related data record for identification for this purpose. These test data are anonymized key data, the key data in turn uniquely assigning the patient-related data record to the patient. In the example embodiment, the patient's name and date of birth are selected as key data from the sensitive patient data during a selection process step 16.

The test data, here the numerical sequence or character string, are then generated from these key data using a one-way hash function and are incorporated in the patient-related data record with the aid of the additional “tag” for identifying the latter. All sensitive patient data contained in the patient-related data record are additionally anonymized in an anonymization process step 20 with the aid of the same one-way hash function and are replaced with numerical sequences or character strings as placeholders. In addition, the key data are incorporated, as test data, in the form of a QR code in each transverse slice, with the result that this QR code is always depicted at the top right-hand edge of the image when displaying a corresponding transverse slice on a monitor. In this case, the corresponding QR code is generated from the key data using a further hash algorithm, a 2-D barcode hash algorithm.

The patient-related data record anonymized in this manner is then delivered from the immediate control area of the hospital to the cloud computing architecture 2 and is stored there in the archive for medical documents during a filing process step 22. If this is the first anonymized patient-related data record for the patient, a new patient file is first of all created in the archive, which file is identified by the test data, that is to say the corresponding numerical sequence or character string. The anonymized patient-related data record is then entered into the newly created patient file. If a patient file containing the corresponding test data already exists, there is no need to create a new patient file and the anonymized patient-related data record is assigned to the patient file containing the test data in the anonymized patient-related data record.

If a doctor is now instructed by the patient to diagnostically evaluate the examination carried out on the computer tomograph 4 in the hospital, the doctor is able to access the archive for medical documents via a client computer 24 which is connected to the cloud computing architecture 2. For this purpose, the doctor starts an application which is locally available on the client computer 24 and which requests the doctor to input the key data relating to the patient, that is to say the patient's name and date of birth, in an input window on the client computer 24. Query data, that is to say a numerical sequence or character string again, are generated by the application on the client computer 24 during a querying process step 26 with the aid of the same one-way hash function which was used to anonymize the sensitive patient data in the patient-related data record in the server station 12 of the hospital. Data records whose test data match the query data or whose numerical sequence or character string matches the numerical sequence or character string generated on the client computer 24 are then searched for in the archive for medical documents in the cloud computing architecture 2.

If corresponding data records are found, the doctor is requested to select a type of illustration from a selection, that is to say a sectional illustration with a specially selected sectional plane or a 3-D illustration of a selected region of the body, for example. The anonymized patient-related data record found is then preprocessed in the cloud computing architecture 2 during a processing process step 28, thus generating display data for display on a monitor. Such preprocessing is, for example, so-called multiplanar reformatting (MRT), also called multiplanar reconstruction, in which sectional illustrations with an arbitrarily selected sectional plane are calculated from the transverse slices, image processing according to the MIP (Maximum Intensity Protection) principle or else a so-called raycasting method. In each case, the QR code contained in each transverse slice is also embedded in the display data.

The display data are then transmitted to the client computer 24 and are double-checked there as part of a comparison process step 30. For this purpose, the key data input by the doctor on the client computer 24 are converted into a QR code with the aid of the abovementioned 2-D barcode hash algorithm and the QR code generated in this manner is compared with the QR code in the display data from the cloud computing architecture 2. If the two QR codes do not match, a security function is triggered, as a result of which the display data are rejected by the client computer 24 and a fault notification consequently appears on the monitor of the client computer 24, which fault notification draws the doctor's attention to the fact that the display data are assigned to an unknown patient.

In contrast, if the QR codes match, the display data are released during a release process step 32 and are displayed as an image on the monitor of the client computer 24. An additional image which is placed over the image based on the display data is also generated during an overlapping process step 34 with the aid of the application locally started on the client computer 24 by the doctor. As a result, the doctor does not see the desired x-ray in which the QR code is depicted at the top right but rather sees the desired x-ray in which the key data are depicted as plain data at the top right, that is to say in which the patient's name and date of birth can be read at the top right, on the monitor of the client computer 24.

The invention is not restricted to the example embodiment described above. Rather, other variants of the invention can also be derived therefrom by a person skilled in the art without departing from the subject matter of the invention. In particular, all individual features described in connection with the example embodiment can furthermore also be combined with one another in another manner without departing from the subject matter of the invention.

1. An image acquisition and processing method for rendering and displaying a medical image based on medical image data for a selected patient, the method comprising:
scanning the selected patient to acquire raw data for the selected patient;
generating the medical image data for the selected patient based on the raw data;
storing the medical image data along with sensitive patient data for the selected patient in a patient-related data record;
extracting the sensitive patient data from the patient-related data record;
generating verification data based on a first portion of the sensitive patient data using a one-way hash function, the first portion of the sensitive patient data uniquely identifying the selected patient;
tagging the patient-related data record with the verification data;
generating an anonymized patient-related data record by anonymizing all of the sensitive patient data in the patient-related data record, the anonymized patient-related data record including the anonymized sensitive patient data and the medical image data;
storing the anonymized patient-related data record on at least one cloud server;
rendering the medical image data stored in the anonymized patient-related data record to generate a rendered medical image for display in response to a received query relating to the sensitive patient data;
applying the one-way hash function to the received query to generate query data;
comparing the query data with the verification data;
displaying the rendered medical image in response to determining that the verification data matches the query data; and
displaying a fault notification in response to determining that the verification data does not match the query data.

2. The method of claim 1, wherein the sensitive patient data is a subset of data stored in the patient-related data record.

3. The method of claim 1, wherein the anonymizing anonymizes all of the sensitive patient data in the patient-related data record based on the one-way hash function.

4. The method of claim 1, wherein the one-way hash function is of type MD5, SHA1 or SHA2.

5. The method of claim 1, wherein the rendering the medical image data is performed on at least one cloud server.

6. The method of claim 1, wherein the rendering the medical image data is performed on a client computer.

7. The method of claim 1, wherein one or more of the displaying of the rendered medical image or the displaying of the fault notification is performed on a client computer.

8. The method of claim 1, wherein the scanning is performed by an imaging modality.

9. The method of claim 1, wherein the displaying a fault notification comprises:
displaying the fault notification without the rendered medical image in response to determining that the verification data does not match the query data.

10. The method of claim 1, further comprising:
rejecting the rendered medical image in response to determining that the verification data does not match the query data; and wherein
the displaying a fault notification displays the fault notification without the rendered medical image.

11. The method of claim 1, further comprising:
dividing the sensitive patient data into the first portion of the sensitive patient data and a second portion of the sensitive patient data.

12. The method of claim 1, wherein the displaying the rendered medical image comprises:
displaying the verification data along with the rendered medical image in response to determining that the verification data matches the query data.

13. The method of claim 12, wherein the verification data is displayed as a 2-D barcode.

14. The method of claim 1, further comprising:
preventing display of the rendered medical image in response to determining that the verification data does not match the query data.

15. The method of claim 1, further comprising:
providing the verification data in the anonymized patient-related data record on a client computer; and wherein
the comparing compares the query data with the verification data at the client computer to determine whether the verification data matches the query data.

16. The method of claim 1, wherein the displaying displays the rendered medical image only in response to determining that the verification data matches the query data.

17. An image acquisition and processing system comprising:
a server station configured to
store medical image data along with sensitive patient data for a selected patient in a patient-related data record, the medical image data for the selected patient generated based on raw data acquired by scanning the selected patient using an imaging modality,
extract the sensitive patient data from the patient-related data record,
generate verification data based on a first portion of the sensitive patient data using a one-way hash function, the first portion of the sensitive patient data uniquely identifying the selected patient,
tag the patient-related data record with the verification data, and
generate an anonymized patient-related data record by anonymizing
all of the sensitive patient data in the patient-related data record, the anonymized patient-related data record including the anonymized sensitive patient data and the medical image data;
a cloud server configured to store the anonymized patient-related data record;
a client computer configured to
apply the one-way hash function to a received query relating to the sensitive patient data to generate query data,
display a rendered medical image generated based on the medical image data stored in the patient-related data record in response to a determination that the verification data matches the query data, and
display a fault notification in response to a determination that the verification data does not match the query data; and
wherein at least one of the cloud server and the client computer are further configured to
render the medical image data stored in the anonymized patient-related data record to generate the rendered medical image, and
compare the query data with the verification data to determine whether the verification data matches the query data.

18. The system of claim 17, wherein the sensitive patient data is a subset of data stored in the patient-related data record.

19. The system of claim 17, wherein the server station is configured to anonymize all of the sensitive patient data in the patient-related data record based on the one-way hash function.

20. The system of claim 17, wherein the one-way hash function is of type MD5, SHA1 or SHA2.

21. The system of claim 17, wherein the client computer is configured to display the fault notification without the rendered medical image in response to the determination that the verification data does not match the query data.

22. The system of claim 17, wherein the client computer is further configured to
reject the rendered medical image in response to the determination that the verification data does not match the query data; and
display the fault notification without the rendered medical image.

23. The system of claim 17, wherein the server station is further configured to divide the sensitive patient data into the first portion of the sensitive patient data and a second portion of the sensitive patient data.

24. The system of claim 17, wherein the client computer is configured to display the verification data along with the rendered medical image in response to the determination that the verification data matches the query data.

25. The system of claim 24, wherein the client computer is further configured to display the verification data as a 2-D barcode.

26. The system of claim 17, wherein the client computer is further configured to prevent display of the rendered medical image in response to the determination that the verification data does not match the query data.

27. The system of claim 17, wherein the client computer is configured to display the rendered medical image only in response to the determination that the verification data matches the query data.
